SEQUENCE LISTING (10 sequences)

SEQ ID NO: 1 - DNA, 5200 bp, Homo sapiens
  CDS (118)..(3699)
  sig_peptide (118)..(183)
gaccacctgatcgaaggaaaaggaaggcacagcggagcgcagagtgagaaccaccaaccgaggcgccgggcagcgacccctgcagcggagacagagactgagcggcccggcaccgccatgcctgcgctctggctgggctgctgcctctgcttctcgctcctcctgcccgcagcccgggccacctccaggagggaagtctgtgattgcaatgggaagtccaggcagtgtatctttgatcgggaacttcacagacaaactggtaatggattccgctgcctcaactgcaatgacaacactgatggcattcactgcgagaagtgcaagaatggcttttaccggcacagagaaagggaccgctgtttgccctgcaattgtaactccaaaggttctcttagtgctcgatgtgacaactctggacggtgcagctgtaaaccaggtgtgacaggagccagatgcgaccgatgtctgccaggcttccacatgctcacggatgcggggtgcacccaagaccagagactgctagactccaagtgtgactgtgacccagctggcatcgcagggccctgtgacgcgggccgctgtgtctgcaagccagctgttactggagaacgctgtgataggtgtcgatcaggttactataatctggatggggggaaccctgagggctgtacccagtgtttctgctatgggcattcagccagctgccgcagctctgcagaatacagtgtccataagatcacctctacctttcatcaagatgttgatggctggaaggctgtccaacgaaatgggtctcctgcaaagctccaatggtcacagcgccatcaagatgtgtttagctcagcccaacgactagatcctgtctattttgtggctcctgccaaatttcttgggaatcaacaggtgagctatgggcaaagcctgtcctttgactaccgtgtggacagaggaggcagacacccatctgcccatgatgtgatcctggaaggtgctggtctacggatcacagctcccttgatgccacttggcaagacactgccttgtgggctcaccaagacttacacattcaggttaaatgagcatccaagcaataattggagcccccagctgagttactttgagtatcgaaggttactgcggaatctcacagccctccgcatccgagctacatatggagaatacagtactgggtacattgacaatgtgaccctgatttcagcccgccctgtctctggagccccagcaccctgggttgaacagtgtatatgtcctgttgggtacaaggggcaattctgccaggattgtgcttctggctacaagagagattcagcgagactggggccttttggcacctgtattccttgtaactgtcaagggggaggggcctgtgatccagacacaggagattgttattcaggggatgagaatcctgacattgagtgtgctgactgcccaattggtttctacaacgatccgcacgacccccgcagctgcaagccatgtccctgtcataacgggttcagctgctcagtgattccggagacggaggaggtggtgtgcaataactgccctcccggggtcaccggtgcccgctgtgagctctgtgctgatggctactttggggacccctttggtgaacatggcccagtgaggccttgtcagccctgtcaatgcaacagcaatgtggaccccagtgcctctgggaattgtgaccggctgacaggcaggtgtttgaagtgtatccacaacacagccggcatctactgcgaccagtgcaaagcaggctacttcggggacccattggctcccaacccagcagacaagtgtcgagcttgcaactgtaaccccatgggctcagagcctgtaggatgtcgaagtgatggcacctgtgtttgcaagccaggatttggtggccccaactgtgagcatggagcattcagctgtccagcttgctataatcaagtgaagattcagatggatcagtttatgcagcagcttcagagaatggaggccctgatttcaaaggctcagggtggtgatggagtagtacctgatacagagctggaaggcaggatgcagcaggctgagcaggcccttcaggacattctgagagatgcccagatttcagaaggtgctagcagatcccttggtctccagttggccaaggtgaggagccaagagaacagctaccagagccgcctggatgacctcaagatgactgtggaaagagttcgggctctgggaagtcagtaccagaaccgagttcgggatactcacaggctcatcactcagatgcagctgagcctggcagaaagtgaagcttccttgggaaacactaacattcctgcctcagaccactacgtggggccaaatggctttaaaagtctggctcaggaggccacaagattagcagaaagccacgttgagtcagccagtaacatggagcaactgacaagggaaactgaggactattccaaacaagccctctcactggtgcgcaaggccctgcatgaaggagtcggaagcggaagcggtagcccggacggtgctgtggtgcaagggcttgtggaaaaattggagaaaaccaagtccctggcccagcagttgacaagggaggccactcaagcggaaattgaagcagataggtcttatcagcacagtctccgcctcctggattcagtgtctccgcttcagggagtcagtgatcagtcctttcaggtggaagaagcaaagaggatcaaacaaaaagcggattcactctcaagcctggtaaccaggcatatggatgagttcaagcgtacacaaaagaatctgggaaactggaaagaagaagcacagcagctcttacagaatggaaaaagtgggagagagaaatcagatcagctgctttcccgtgccaatcttgctaaaagcagagcacaagaagcactgagtatgggcaatgccactttttatgaagttgagagcatccttaaaaacctcagagagtttgacctgcaggtggacaacagaaaagcagaagctgaagaagccatgaagagactctcctacatcagccagaaggtttcagatgccagtgacaagacccagcaagcagaaagagccctggggagcgctgctgctgatgcacagagggcaaagaatggggccggggaggccctggaaatctccagtgagattgaacaggagattgggagtctgaacttggaagccaatgtgacagcagatggagccttggccatggaaaagggactggcctctctgaagagtgagatgagggaagtggaaggagagctggaaaggaaggagctggagtttgacacgaatatggatgcagtacagatggtgattacagaagcccagaaggttgataccagagccaagaacgctggggttacaatccaagacacactcaacacattagacggcctcctgcatctgatggaccagcctctcagtgtagatgaagaggggctggtcttactggagcagaagctttcccgagccaagacccagatcaacagccaactgcggcccatgatgtcagagctggaagagagggcacgtcagcagaggggccacctccatttgctggagacaagcatagatgggattctggctgatgtgaagaacttggagaacattagggacaacctgcccccaggctgctacaatacccaggctcttgagcaacagtgaagctgccataaatatttctcaactgaggttcttgggatacagatctcagggctcgggagccatgtcatgtgagtgggtgggatggggacatttgaacatgtttaatgggtatgctcaggtcaactgacctgaccccattcctgatcccatggccaggtggttgtcttattgcaccatactccttgcttcctgatgctgggcatgaggcagataggcactggtgtgagaatgatcaaggatctggaccccaaagatagactggatggaaagacaaactgcacaggcagatgtttgcctcataatagtcgtaagtggagtcctggaatttggacaagtgctgttgggatatagtcaacttattctttgagtaatgtgactaaaggaaaaaactttgactttgcccaggcatgaaattcttcctaatgtcagaacagagtgcaacccagtcacactgtggccagtaaaatactattgcctcatattgtcctctgcaagcttcttgctgatcagagttcctcctacttacaacccagggtgtgaacatgttctccattttcaagctggaagaagtgagcagtgttggagtgaggacctgtaaggcaggcccattcagagctatggtgcttgctggtgcctgccaccttcaagttctggacctgggcatgacatcctttcttttaatgatgccatggcaacttagagattgcatttttattaaagcatttcctaccagcaaagcaaatgttgggaaagtatttactttttcggtttcaaagtgatagaaaagtgtggcttgggcattgaaagaggtaaaattctctagatttattagtcctaattcaatcctacttttcgaacaccaaaaatgatgcgcatcaatgtattttatcttattttctcaatctcctctctctttcctccacccataataagagaatgttcctactcacacttcagctgggtcacatccatccctccattcatccttccatccatctttccatccattacctccatccatccttccaacatatatttattgagtacctactgtgtgccaggggctggtgggacagtggtgacatagtctctgccctcatagagttgattgtctagtgaggaagacaagcatttttaaaaaataaatttaaacttacaaactttgtttgtcacaagtggtgtttattgcaataaccgcttggtttgcaacctctttgctcaacagaacatatgttgcaagaccctcccatgggcactgagtttggcaaggatgacagagctctgggttgtgcacatttctttgcattccagcgtcactctgtgccttctacaactgattgcaacagactgttgagttatgataacaccagtgggaattgctggaggaaccagaggcacttccaccttggctgggaagactatggtgctgccttgcttctgtatttccttggattttcctgaaagtgtttttaaataaagaacaattgttagatgccaaaaa

SEQ ID NO: 2 - protein, 1193 aa, Homo sapiens [sequence omitted]

SEQ ID NO: 3 - DNA, 4316 bp, Homo sapiens
  sig_peptide (118)..(183)
  CDS (118)..(3453)
  repeat_unit (4021)..(4316)
  polyA_site (4296)..(4316)
gaccacctgatcgaaggaaaaggaaggcacagcggagcgcagagtgagaaccaccaaccgaggcgccgggcagcgacccctgcagcggagacagagactgagcggcccggcaccgccatgcctgcgctctggctgggctgctgcctctgcttctcgctcctcctgcccgcagcccgggccacctccaggagggaagtctgtgattgcaatgggaagtccaggcagtgtatctttgatcgggaacttcacagacaaactggtaatggattccgctgcctcaactgcaatgacaacactgatggcattcactgcgagaagtgcaagaatggcttttaccggcacagagaaagggaccgctgtttgccctgcaattgtaactccaaaggttctcttagtgctcgatgtgacaactctggacggtgcagctgtaaaccaggtgtgacaggagccagatgcgaccgatgtctgccaggcttccacatgctcacggatgcggggtgcacccaagaccagagactgctagactccaagtgtgactgtgacccagctggcatcgcagggccctgtgacgcgggccgctgtgtctgcaagccagctgttactggagaacgctgtgataggtgtcgatcaggttactataatctggatggggggaaccctgagggctgtacccagtgtttctgctatgggcattcagccagctgccgcagctctgcagaatacagtgtccataagatcacctctacctttcatcaagatgttgatggctggaaggctgtccaacgaaatgggtctcctgcaaagctccaatggtcacagcgccatcaagatgtgtttagctcagcccaacgactagatcctgtctattttgtggctcctgccaaatttcttgggaatcaacaggtgagctatgggcaaagcctgtcctttgactaccgtgtggacagaggaggcagacacccatctgcccatgatgtgatcctggaaggtgctggtctacggatcacagctcccttgatgccacttggcaagacactgccttgtgggctcaccaagacttacacattcaggttaaatgagcatccaagcaataattggagcccccagctgagttactttgagtatcgaaggttactgcggaatctcacagccctccgcatccgagctacatatggagaatacagtactgggtacattgacaatgtgaccctgatttcagcccgccctgtctctggagccccagcaccctgggttgaacagtgtatatgtcctgttgggtacaaggggcaattctgccaggattgtgcttctggctacaagagagattcagcgagactggggccttttggcacctgtattccttgtaactgtcaagggggaggggcctgtgatccagacacaggagattgttattcaggggatgagaatcctgacattgagtgtgctgactgcccaattggtttctacaacgatccgcacgacccccgcagctgcaagccatgtccctgtcataacgggttcagctgctcagtgattccggagacggaggaggtggtgtgcaataactgccctcccggggtcaccggtgcccgctgtgagctctgtgctgatggctactttggggacccctttggtgaacatggcccagtgaggccttgtcagccctgtcaatgcaacagcaatgtggaccccagtgcctctgggaattgtgaccggctgacaggcaggtgtttgaagtgtatccacaacacagccggcatctactgcgaccagtgcaaagcaggctacttcggggacccattggctcccaacccagcagacaagtgtcgagcttgcaactgtaaccccatgggctcagagcctgtaggatgtcgaagtgatggcacctgtgtttgcaagccaggatttggtggccccaactgtgagcatggagcattcagctgtccagcttgctataatcaagtgaagattcagatggatcagtttatgcagcagcttcagagaatggaggccctgatttcaaaggctcagggtggtgatggagtagtacctgatacagagctggaaggcaggatgcagcaggctgagcaggcccttcaggacattctgagagatgcccagatttcagaaggtgctagcagatcccttggtctccagttggccaaggtgaggagccaagagaacagctaccagagccgcctggatgacctcaagatgactgtggaaagagttcgggctctgggaagtcagtaccagaaccgagttcgggatactcacaggctcatcactcagatgcagctgagcctggcagaaagtgaagcttccttgggaaacactaacattcctgcctcagaccactacgtggggccaaatggctttaaaagtctggctcaggaggccacaagattagcagaaagccacgttgagtcagccagtaacatggagcaactgacaagggaaactgaggactattccaaacaagccctctcactggtgcgcaaggccctgcatgaaggagtcggaagcggaagcggtagcccggacggtgctgtggtgcaagggcttgtggaaaaattggagaaaaccaagtccctggcccagcagttgacaagggaggccactcaagcggaaattgaagcagataggtcttatcagcacagtctccgcctcctggattcagtgtctccgcttcagggagtcagtgatcagtcctttcaggtggaagaagcaaagaggatcaaacaaaaagcggattcactctcaagcctggtaaccaggcatatggatgagttcaagcgtacacaaaagaatctgggaaactggaaagaagaagcacagcagctcttacagaatggaaaaagtgggagagagaaatcagatcagctgctttcccgtgccaatcttgctaaaagcagagcacaagaagcactgagtatgggcaatgccactttttatgaagttgagagcatccttaaaaacctcagagagtttgacctgcaggtggacaacagaaaagcagaagctgaagaagccatgaagagactctcctacatcagccagaaggtttcagatgccagtgacaagacccagcaagcagaaagagccctggggagcgctgctgctgatgcacagagggcaaagaatggggccggggaggccctggaaatctccagtgagattgaacaggagattgggagtctgaacttggaagccaatgtgacagcagatggagccttggccatggaaaagggactggcctctctgaagagtgagatgagggaagtggaaggagagctggaaaggaaggagctggagtttgacacgaatatggatgcagtacagatggtgattacagaagcccagaaggttgataccagagccaagaacgctggggttacaatccaagacacactcaacacattagacggcctcctgcatctgatgggtatgtgaacccacaacccacaaccttccagctccatgctccagggctttgctccagaacactcactatacctagccccagcaaaggggagtctcagctttccttaaggatatcagtaaatgtgctttgtttccaggcccagataactttcggcaggttcccttacatttactggaccctgttttaccgttgctaagatgggtcactgaacacctattgcacttgggggtaaaggtctgtgggccaaagaacaggtgtatataagcaacttcacagaacacgagacagcttgggaatcctgctaaagagtctggcctggaccctgagaagccagtggacagttttaagcagaggaataacatcaccactgtatatttcagaaagatcactagggcagccgagtggaggaaagcttgaagagggggttagagagaaggcaggttgagactacttaagatattgttgaaataattgaagagagaaatgacaggagcctgctctaaggcagtagaatggtggctgggaagatgtgaaggaagattttcccagtctgtgaagtcaagaatcacttgccggccgggtgtggtggctcacgcctgtaattctagcactttgggagactgaagcgggtggatcacccgaggtcaggagttgaagaccagcctggccaacatggtgaaaccctgtctctactaaaagtacaaaaattagctggatgatggtggtgggcgcctgtaattccagctactcaggagtctgaggcaggagaatcgcttgaacccaggaggcgaggttacagtgagccaagattgcaccactgctcttccagcctgggaacagagagactgcctaaaaaaaaaaaaaaaaaaaa

SEQ ID NO: 4 - protein, 1111 aa, Homo sapiens [sequence omitted]

SEQ ID NO: 5 - DNA, 530 bp, Homo sapiens
  misc_feature: Portion of Domain III of laminin gamma 2
aattctgccaggattgtgcttctggctacaagagagattcagcgagactggggccttttggcacctgtattccttgtaactgtcaagggggaggggcctgtgatccagacacaggagattgttattcaggggatgagaatcctgacattgagtgtgctgactgcccaattggtttctacaacgatccgcacgacccccgcagctgcaagccatgtccctgtcataacgggttcagctgctcagtgattccggagacggaggaggtggtgtgcaataactgccctcccggggtcaccggtgcccgctgtgagctctgtgctgatggctactttggggacccctttggtgaacatggcccagtgaggccttgtcagccctgtcaatgcaacagcaatgtggaccccagtgcctctgggaattgtgaccggctgacaggcaggtgtttgaagtgtatccacaacacagccggcatctactgcgaccagtgcaaagcaggctacttcggggacccattggctcccaacccagca

SEQ ID NO: 6 - protein, 177 aa, Homo sapiens
  misc_feature: Portion of Domain III of laminin gamma 2.
QFCQDCASGYKRDSARLGPFGTCIPCNCQGGGACDPDTGDCYSGDENPDIECADCPIGFYNDPHDPRSCKPCPCHNGFSCSVMPETEEVVCNNCPPGVTGARCELCADGYFGDPFGEHGPVRPCQPCQCNNNVDPSASGNCDRLTGRCLKCIHNTAGIYCDQCKAGYFGDPLAPNPA

SEQ ID NO: 7 - DNA, 681 bp, Homo sapiens
  misc_feature: Complete domain III of laminin gamma 2
tgtatatgtcctgttgggtacaaggggcaattctgccaggattgtgcttctggctacaagagagattcagcgagactggggccttttggcacctgtattccttgtaactgtcaagggggaggggcctgtgatccagacacaggagattgttattcaggggatgagaatcctgacattgagtgtgctgactgcccaattggtttctacaacgatccgcacgacccccgcagctgcaagccatgtccctgtcataacgggttcagctgctcagtgattccggagacggaggaggtggtgtgcaataactgccctcccggggtcaccggtgcccgctgtgagctctgtgctgatggctactttggggacccctttggtgaacatggcccagtgaggccttgtcagccctgtcaatgcaacagcaatgtggaccccagtgcctctgggaattgtgaccggctgacaggcaggtgtttgaagtgtatccacaacacagccggcatctactgcgaccagtgcaaagcaggctacttcggggacccattggctcccaacccagcagacaagtgtcgagcttgcaactgtaaccccatgggctcagagcctgtaggatgtcgaagtgatggcacctgtgtttgcaagccaggatttggtggccccaactgtgagcatggagcattcagc

SEQ ID NO: 8 - protein, 227 aa, Homo sapiens
  misc_feature: Complete domain III of laminin gamma 2.
CICPVGYKGQFCQDCASGYKRDSARLGPFGTCIPCNCQGGGACDPDTGDCYSGDENPDIECADCPIGFYNDPHDPRSCKPCPCHNGFSCSVMPETEEVVCNNCPPGVTGARCELCADGYFGDPFGEHGPVRPCQPCQCNNNVDPSASGNCDRLTGRCLKCIHNTAGIYCDQCKAGYFGDPLAPNPADKCRACNCNPMGSEPVGCRSDGTCVCKPGFGGPNCEHGAFS

SEQ ID NO: 9 - protein, 9 aa, Homo sapiens
  misc_feature: N-terminal portion of domain III of laminin gamma 2.
CICPVGYKG

SEQ ID NO: 10 - protein, 41 aa, Homo sapiens
  misc_feature: C-terminal portion of domain III of laminin gamma 2.
DKCRACNCNPMGSEPVGCRSDGTCVCKPGFGGPNCEHGAFS

(19) United States
(12) Patent Application Publication
(10) Pub. No.: US 2007/0202111 A1
(43) Pub. Date: Aug. 30, 2007

(54) Use of antibodies to the gamma 2 chain of laminin 5 to inhibit tumor growth and metastasis

(75) Inventors: Karl Tryggvason, Stockholm (SE); Sirpa Salo, Oulu (FI)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: BioStratum, Inc.

(21) Appl. No.: 11/510,314

(22) Filed: Aug. 25, 2006

Related U.S. Application Data
(63) Continuation of application No. 10/695,559, filed on Oct. 28, 2003, now abandoned.
Application No. 10/695,559 is a continuation of application No. 09/756,071, filed on Jan. 8, 2001, now Pat. No. 6,955,924.
(60) Provisional application No. 60/422,009, filed on Oct. 29, 2002.

(51) Int. Cl. A61K 39/395; C07K 16/30; C12N 5/16

(52) U.S. Cl. 424/155.1; 435/344; 530/388.8

(57) ABSTRACT

The present invention provides a methods and compositions for inhibiting tumor growth and/or metastasis involving the administering to a subject with a laminin 5-secreting tumor of an amount effective to inhibit tumor growth and/or metastasis of an antibody that binds to one or more epitopes of the laminin 5 γ2 chain.

### CROSS REFERENCE

This application claims priority to U.S. Provisional Patent Application 60/422,009 filed Oct. 29, 2002, and is a continuation in part of U.S. patent application Ser. No. 09/756,071 filed Jan. 8, 2001, which are herein incorporated by reference in their entirety.

### BACKGROUND OF THE INVENTION

Laminins are basement membrane glycoproteins with diverse biological functions including cell adhesion, proliferation, migration and differentiation. Thus far, 11 genetically distinct chains forming at least 12 laminin isoforms have been characterized. Every member of this growing protein family has a heterotrimeric chain composition of α, β, and γ chains that are formed through an intracellular self-assembly mechanism.

Laminin-5 is a specific component of epithelial basement membranes with the chain composition α3β3γ2 (Kallunki, et al., J. Cell Biol. 119: 679-93, 1992). The γ2 chain has a mass of ≈130 kd and is thus smaller than the “classical” ≈200 kd β1 and γ1 light chains of laminin 1. Expression of laminin 5 chains is often up-regulated in epithelial cancers, such as squamous cell carcinomas and gastric carcinomas, but not in mesenchymally derived cancers (Larjava, et al., J. Clin. Invest. 92: 1425-35, 1993) (Pyke, et al., Am. J. Pathol. 145: 782-91, 1994) (Pyke, et al., Cancer Res. 55: 4132-9, 1995) (Tani, et al., Am. J. Pathol. 149: 781-93, 1996) (Orian-Rousseau, et al., J. Cell. Sci. 111: 19932004, 1998) (Sordat, et al., J. Pathol. 185: 44-52, 1998). However, down-regulation has been reported in epithelial prostate and breast carcinomas (Hao, J., Yang, Am. J. Pathol. 149: 1341-9, 1996) (Martin, et al., Mol. Med. 4: 602-613, 1998). In colon adenocarcinomas, both gene and protein expression of the γ2 chain seem to be a characteristic of cancer cells with a budding phenotype (Larjava, et al., J. Clin. Invest. 92: 1425-35, 1993) (Pyke, et al., Am. J. Pathol. 145: 782-91, 1994) (Pyke, et al., Cancer Res. 55: 4132-9, 1995). Tumor cell budding in colorectal carcinoma has also been associated with the presence of intracellular laminin-5 (Sordat, et al., J. Pathol. 185: 44-52, 1998).

The γ2 chain of laminin-5 has also been shown to be strongly expressed in malignant cells located at the invasion front of several human carcinomas, as determined by in situ hybridization and immunohistochemical staining (Pyke, C., Romer, J., Kallunki, P., Lund, L. R., Ralfkiaer, E., Dano, K. & Tryggvason, K. (1994) Am. J. Pathol. 145: 782-791; Pyke, C., Salo, S., Ralfkiaer, E., Romer, J., Dano, K. & Tryggvason, K. (1995) Cancer Res. 55: 4132-4139). However, no studies have shown that antibodies to the γ2 chain of laminin 5 can be used to inhibit tumor cell growth.

### SUMMARY OF THE INVENTION

The present invention provides antibodies, compositions and methods for inhibiting tumor growth and/or metastasis. In one aspect, the present invention provides antibodies that bind to one or more epitopes of domain III of the human laminin 5 γ2 chain (SEQ ID NOS: 2 and 4).

In another aspect, the present invention provides a method for inhibiting tumor growth and/or metastasis comprising administering to a subject with a laminin 5-secreting tumor an amount effective to inhibit tumor growth and/or metastasis of an antibody that binds to one or more epitopes of the laminin 5 γ2 chain. In one embodiment, the antibody binds to one or more epitopes of domain III of the laminin 5 γ2 chain.

In a further aspect, the present invention provides a pharmaceutical composition comprising an antibody that binds to the laminin 5 γ2 chain and one or more further anti-tumor agents. In various embodiments of this aspect, the antibody is selective for one or more epitopes in domain III of the laminin 5 γ2 chain, and/or the further anti-tumor agent is a chemotherapeutic.

### BRIEF DESCRIPTION OF THE DRAWINGS

FIG. 1 shows efficiency of human laminin-5 and recombinant human laminin γ2 chain for attachment of HaCat keratinocytes and KLN205 squamous carcinoma cells in vitro. The attachment efficiency was compared with the efficiency with which the cells bound to laminin-1. Substrate concentrations (10 μg/ml) providing maximum attachment to laminin-1 and laminin-5 were used. The results are presented as means+/−SD calculated from at least four duplicate series; the values for laminin-1 were given the arbitrary value of 100%.

FIGS. 2A-B show the effects of polyclonal γ2 chain antibodies on the migration of KLN205 squamous carcinoma cells in Boyden and Transwell chamber assays of migration.

FIG. 3 shows tumor growth inhibition using Mab 5D5 and CPT-11 on day 31 in the HT29-e28 cell line.

FIGS. 4A-E show tumor growth curves for individual mice in the HT29-e28 study.

### DETAILED DESCRIPTION OF THE INVENTION

In one aspect, the present invention provides a method for inhibiting tumor growth and/or metastasis comprising administering to a subject with a laminin 5-secreting tumor an amount effective to inhibit tumor growth and/or metastasis of an antibody that binds to one or more epitopes of the laminin 5 γ2 chain. In a preferred embodiment, the subject is a mammal; in a more preferred embodiment, the subject is human.

As used herein, the term “inhibiting tumor growth” means to reduce the amount of tumor growth that would occur in the absence of treatment, and includes decrease in tumor size and/or decrease in the rate of tumor growth.

As used herein, the term “inhibiting tumor metastasis” means to reduce the amount of tumor metastasis that would occur in the absence of treatment, and includes decrease in the number and/or size of metastases.

As used herein, the term “laminin-5 secreting tumor” means a tumor that expresses detectable amounts of laminin 5. Such tumors include, but are not limited to, carcinomas. Such carcinomas include, but are not limited to squamous cell carcinomas (including but not limited to squamous cell carcinoma of skin, cervix, and vulva), gastric carcinomas, colon adenocarcinomas, colorectal carcinomas, and cervical carcinomas.

As used herein, the term “laminin 5 γ2 chain” preferably refers to the human laminin 5 γ2 chain, with protein sequences comprising the amino acid sequence of SEQ ID NO:2 or SEQ ID NO:4, and derivatives thereof.

As used herein, the term “epitope” refers to a specific site within the protein that is bound by the antibody, which includes both linear and non-linear epitopes.

In a preferred embodiment, the antibody binds to one or more epitopes of domain III of the laminin 5 γ2 chain. As used herein, the term “domain III of the laminin 5 γ2 chain” refers to a 177 amino acid region of SEQ ID NO:2 between residues 391 and 567 (Kallunki et al., J. Cell Biol. 119:679-693 (1992)), which is presented herein as SEQ ID NO:8. In a further preferred embodiment, the antibody binds to one or more epitopes within domain III that are contained within the amino acid sequence of SEQ ID NO:6 and does not bind to epitopes within domain III that are within the amino acid sequence of SEQ ID NOS: 9 and 10.

The antibody can be a polyclonal antibody or a monoclonal antibody, but preferably is a monoclonal antibody. The antibodies can be humanized, fully human, or murine forms of the antibodies.

In another embodiment, the methods of the invention can be used in combination with surgery on the subject, wherein surgery includes primary surgery for removing one or more tumors, secondary cytoreductive surgery, and palliative secondary surgery.

In a further embodiment, the methods of the invention further comprises treating the subject with chemotherapy and/or radiation therapy. One benefit of such a method if that use of the antibody permits a reduction in the chemotherapy and/or radiation dosage necessary to inhibit tumor growth and/or metastasis. As used herein, “radiotherapy” includes but is not limited to the use of radio-labeled compounds targeting tumor cells. Any reduction in chemotherapeutic or radiation dosage benefits the patient by resulting in fewer and decreased side effects relative to standard chemotherapy and/or radiation therapy treatment.

In this embodiment, the antibody may be administered prior to, at the time of, or shortly after a given round of treatment with chemotherapeutic and/or radiation therapy. In a preferred embodiment, the antibody is administered prior to or simultaneously with a given round of chemotherapy and/or radiation therapy. In a most preferred embodiment, the antibody is administered prior to or simultaneously with each round of chemotherapy and/or radiation therapy. The exact timing of antibody administration will be determined by an attending physician based on a number of factors, but the antibody is generally administered between 24 hours before a given round of chemotherapy and/or radiation therapy and simultaneously with a given round of chemotherapy and/or radiation therapy.

The methods of the invention are appropriate for use with chemotherapy using one or more cytotoxic agent (ie: chemotherapeutic), including, but not limited to, cyclophosphamide, taxol, 5-fluorouracil, adriamycin, cisplatinum, methotrexate, cytosine arabinoside, mitomycin C, prednisone, vindesine, carbaplatinum, and vincristine. The cytotoxic agent can also be an antiviral compound which is capable of destroying proliferating cells. For a general discussion of cytotoxic agents used in chemotherapy, see Sathe, M. et al., Cancer Chemotherapeutic Agents: Handbook of Clinical Data (1978), hereby incorporated by reference.

The methods of the invention are also particularly suitable for those patients in need of repeated or high doses of chemotherapy and/or radiation therapy.

In practicing the invention, the amount or dosage range of antibody employed is one that effectively inhibits tumor growth and/or metastasis. The actual dosage range is based on a variety of factors, including the age, weight, sex, medical condition of the individual, the severity of the condition, and the route of administration. An inhibiting amount of antibody that can be employed ranges generally between 0.01 μg/kg body weight and 15 mg/kg body weight, preferably ranging between 0.05 μg/kg and 10 mg/kg body weight, more preferably between 1 μg/kg and 10 mg/kg body weight, and even more preferably between about 10 μg/kg and 5 mg/kg body weight.

The antibody may be administered by any suitable route, but is preferably administered parenterally in dosage unit formulations containing conventional pharmaceutically acceptable carriers, adjuvants, and vehicles. The term “parenteral” as used herein includes, subcutaneous, intravenous, intraarterial, intramuscular, intrasternal, intratendinous, intraspinal, intracranial, intrathoracic, infusion techniques or intraperitoneally. In preferred embodiments, antibody is administered intravenously or subcutaneously.

The antibody may be made up in a solid form (including granules, powders or suppositories) or in a liquid form (e.g., solutions, suspensions, or emulsions). Antibody may be applied in a variety of solutions. Suitable solutions for use in accordance with the invention are sterile, dissolve sufficient amounts of the antibody, and are not harmful for the proposed application.

The antibody may be subjected to conventional pharmaceutical operations such as sterilization and/or may contain conventional adjuvants, such as preservatives, stabilizers, wetting agents, emulsifiers, buffers etc.

For administration, the antibody is ordinarily combined with one or more adjuvants appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, the antibody may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

In another aspect, the present invention provides isolated antibodies that bind to one or more epitopes of domain III of the laminin 5 γ2 chain, hybridoma cells that produce isolated monoclonal antibodies, and pharmaceutical compositions comprising such monoclonals. In a further preferred embodiment, the isolated antibody binds to one or more epitopes within the amino acid sequence of SEQ ID NO:6 and does not bind to epitopes within the amino acid sequence of SEQ ID NOS: 9 and 10. The isolated antibody can be a polyclonal antibody or a monoclonal antibody, but preferably is a monoclonal antibody. In a further embodiment, the isolated antibodies are humanized. In a further embodiment, the isolated antibody is prepared as a pharmaceutical composition, combined with one or more appropriate pharmaceutical carriers, as described above.

These isolated antibodies are useful in all of the methods of the invention, as well as in diagnostic use for detecting the presence of invasive cells in a tissue sample. In a preferred embodiment, diagnostic use of the isolated antibodies of the invention comprises contacting a tumor tissue with one or more isolated antibodies to form an immunocomplex, and detecting formation of the immunocomplex, wherein the formation of the immunocomplex correlates with the presence of invasive cells in the tissue. The contacting can be performed in vivo, using labeled isolated antibodies and standard imaging techniques, or can be performed in vitro on tissue samples.

In a preferred embodiment, the tissue is a tumor tissue. In a further preferred embodiment, the tumor tissue is a laminin 5 secreting tumor tissue. More preferably, the tumor tissue is a carcinoma, including but are not limited to squamous cell carcinomas (including but not limited to squamous cell carcinoma of skin, cervix, and vulva), gastric carcinomas, colon adenocarcinomas, colorectal carcinomas, and cervical carcinomas.

In a further preferred embodiment of this aspect of the invention, the isolated monoclonal antibodies are of the IgG isotype. In a further preferred embodiment, the isolated monoclonal antibodies are selected from the group consisting of those designated herein as 4G1, 5D5 and 6C12, and the hybridomas expressing these monoclonals, which are deposited with the American Type Tissue Collection as ATCC accession numbers ______, ______, and ______. A more detailed description of the production of these particular hybridomas and monoclonal antibodies, and their use, is provided below.

The additional components of pharmaceutical compositions comprising one or more of these isolated antibodies are as described above.

Antibodies can be made by well-known methods, such as described in Harlow and Lane, Antibodies; A Laboratory Manual, Cold Spring Harbor Laboratory, Cold Spring Harbor, N.Y., (1988). In one example, pre-immune serum is collected prior to the first immunization. A peptide portion of the amino acid sequence of a laminin 5 γ2 chain polypeptide, together with an appropriate adjuvant, is injected into an animal in an amount and at intervals sufficient to elicit an immune response. Animals are bled at regular intervals, preferably weekly, to determine antibody titer. The animals may or may not receive booster injections following the initial immunization. At about 7 days after each booster immunization, or about weekly after a single immunization, the animals are bled, the serum collected, and aliquots are stored at about −20° C. Polyclonal antibodies against the laminin 5 γ2 chain polypeptides can then be purified directly by passing serum collected from the animal through a column to which non-antigen-related proteins prepared from the same expression system without the laminin 5 γ2 chain polypeptides bound.

Monoclonal antibodies can be produced by obtaining spleen cells from the animal. (See Kohler and Milstein, Nature 256, 495-497 (1975)). In one example, monoclonal antibodies (mAb) of interest are prepared by immunizing inbred mice with a laminin 5 γ2 chain polypeptide, or portion thereof. The mice are immunized by the IP or SC route in an amount and at intervals sufficient to elicit an immune response. The mice receive an initial immunization on day 0 and are rested for about 3 to about 30 weeks. Immunized mice are given one or more booster immunizations of by the intravenous (IV) route. Lymphocytes from antibody positive mice are obtained by removing spleens from immunized mice by standard procedures known in the art. Hybridoma cells are produced by mixing the splenic lymphocytes with an appropriate fusion partner under conditions that allow formation of stable hybridomas. The antibody producing cells and fusion partner cells are fused in polyethylene glycol at concentrations from about 30% to about 50%. Fused hybridoma cells are selected by growth in hypoxanthine, thymidine and aminopterin supplemented Dulbecco's Modified Eagles Medium (DMEM) by procedures known in the art. Supernatant fluids are collected from growth positive wells and are screened for antibody production by an immunoassay such as solid phase immunoradioassay. Hybridoma cells from antibody positive wells are cloned by a technique such as the soft agar technique of MacPherson, Soft Agar Techniques, in Tissue Culture Methods and Applications, Kruse and Paterson, Eds., Academic Press, 1973.

To generate such an antibody response, a laminin 5 γ2 chain polypeptide or portion thereof is typically formulated with a pharmaceutically acceptable carrier for parenteral administration. Such acceptable adjuvants include, but are not limited to, Freund's complete, Freund's incomplete, alum-precipitate, water in oil emulsion containing Corynebacterium parvum and tRNA. The formulation of such compositions, including the concentration of the polypeptide and the selection of the vehicle and other components, is within the knowledge of those of skill of the art.

The term antibody as used herein is intended to include antibody fragments thereof which are selectively reactive with the laminin 5 γ2 chain polypeptides. Antibodies can be fragmented using conventional techniques, and the fragments screened for utility in the same manner as described above for whole antibodies. For example, F(ab′)₂ fragments can be generated by treating antibody with pepsin. The resulting F(ab′)₂ fragment can be treated to reduce disulfide bridges to produce Fab′ fragments.

In another aspect, the present invention provides pharmaceutical compositions comprising an antibody that binds to the laminin 5 γ2 chain and one or more further anti-tumor agent. In a preferred embodiment of this aspect of the invention, the antibody binds to one or more epitopes in domain III of the laminin 5 γ2 chain, as described above. In a further preferred embodiment, the isolated antibody binds to one or more epitopes within the amino acid sequence of SEQ ID NO:6 and does not bind to epitopes within the amino acid sequence of SEQ ID NOS: 9 and 10. The antibody can be a polyclonal antibody or a monoclonal antibody, but preferably is a monoclonal antibody. In a further preferred embodiment of this aspect of the invention, the further anti-tumor agent is a chemotherapeutic agent, such as one or more of those described above. The components of the pharmaceutical composition may be pre-mixed together or may be combined at any time prior to administration to a patient in need thereof.

The examples below are meant by way of illustration, and are not meant to be limiting as to the scope of the instant disclosure.

### EXAMPLE 1

The following example demonstrates the effect of laminin-5, including the γ2 chain of laminin-5, on cell adhesion and cell migration.

Materials and Methods

Cells and Cell Culture

A mouse squamous cell carcinoma cell line, KLN205 (cat. no. ATCC CRL-1453), was obtained from American Type Culture Collection (Rockville, Md.). The cells were maintained as monolayer cultures in Eagle's minimum essential medium (MEM) containing non-essential amino acids and Earle's BSS supplemented with 10% fetal calf serum (FCS). The HaCat human keratinocyte cell line was a kind gift from Dr. Fuzenig (Heidelberg, Germany). The HaCat cells were cultured in Dulbecco's MEM supplemented with 10% FCS. However, when the cells were cultured for the production of laminin-5, the medium was replaced by serum-free medium.

Preparation of Proteins

Mouse EHS laminin (laminin-1) was obtained from GIBCO BRL. Fibronectin was purified from FCS using a gelatin-Sepharose 4B column (Sigma) as described in Vuento, M. & Vaheri, A. (1979) Biochem. J. 183: 331-337.34 and Gillies, R. J., Didier, N. & Denton, M. (1986) Anal. Biochem. 159: 109-113. Human laminin-5 was immunoaffinity purified from the media of HaCat cells cultured for three days in the absence of serum. Briefly, the medium was first passed through a 5 ml gelatin-Sepharose column (Sigma, St. Louis, Mo.) to ensure the complete absence of fibronectin from the protein Separation, after which the medium was passed through a 10 ml anti-laminin γ2-Sepharose affinity column in order to bind laminin-5 molecules. Both columns were equilibrated in phosphate-buffered saline. The anti-laminin γ2Sepharose affinity column was prepared by coupling a Protein A-purified anti-γ2 IgG (8 mg/ml) to 10 ml of CNBr-activated Sepharose (Pharmacia, Uppsala, Sweden). The anti-γ2 IgG was purified from a rabbit polyclonal antiserum prepared against a GST-fusion protein containing domain III of the γ2 chain (Pyke, C., Salo, S., Ralfkiaer, E., Romer, J., Dano, K. & Tryggvason, K. (1995) Cancer Res. 55: 4132-4139). The laminin-5 was eluted from the immunoaffinity column using 50 mM triethanolamine, pH 11.25, 0.1% Triton X-100 and neutralized directly with 1 M Tris-HCl, pH 7.0. Collected fractions were analyzed by SDS-PAGE and Western blotting using the same polyclonal antibodies as used for the preparation of the affinity column. Fractions containing laminin-5 were pooled and dialyzed against 50 mM Tris-HCl, 0,1 M NaCl, pH 7.4. Some batches of laminin-5 were denatured with 5 M urea and renatured to study the effects of the treatment on adhesion and migration properties.

Generation of Recombinant Baculovirus and Expression of Recombinant Laminin 2 Chain

The γ2 chain of laminin-5 was expressed as recombinant protein using the baculovirus system and purified for studies on its functional properties. A full-length human laminin γ2 chain cDNA containing 6 bp of the 5′ UTR and 822 bp of the 3′ UTR was constructed from four overlapping cDNA clones L52, HT2-7, L15 and L61 (Kallunki, P., Sainio, K., Eddy, R., Byers, M., Kallunki, T., Sariola, H., Beck, K., Hirvonen, H., Shows, T. B. & Tryggvason, K. (1992) J. Cell Biol. 119: 679-693). The resulting 4,402 bp cDNA was analyzed by restriction enzyme mapping and partial sequencing, and cloned into the pVL1393 recombinant transfer plasmid prior to transfer into the AcNPV-γ2 baculovirus vector kindly provided by Max Summers (Texas A&M University). This baculovirus vector containing the human laminin γ2 chain cDNA under the transcriptional control of the polyhedrin promoter was produced and purified following standard procedures, except that it was first enriched according to the method of Pen, et al. (Pen, J., Welling, G. W. & Welling-Wester, S. (1989), Nucl. Acid. Res. 17: 451) from the virus containing medium obtained by co-transfecting Sf9 cells with the wild-type virus (AcNPV) DNA and the recombinant transfer vector pVL 1393-γ2. For expression of the recombinant protein, High Five (H5) cells were infected with the recombinant virus at a multiplicity of infection (MOI) of 5-10 pfu per cell by using standard protocols.

The recombinant γ2 chain was purified by first resuspending the cells in 10 volumes of 50 mM Tris-HCl, pH 7.4, 100 mM NaCl, 2.5 mM EDTA, 1% Triton X-100, 1 mM PMSF and 1 mM NEM followed by homogenization in a Dounce homogenizer. The protein was extracted for 60 minutes on ice and solubilized proteins were removed by centrifugation at 1500×g for 10 minutes at 4° C. The pellet was extracted again with buffer containing 1-3 M urea. The recombinant γ2 chain was extracted with a buffer containing 5 M urea, and renatured by dialysis against 50 mM Tris-HCl, pH 7.4, 100 mM NaCl.

Preparation of Antibodies

Polyclonal antiserum against domain III of the laminin γ2 chain was prepared and characterized as described previously. Briefly, rabbits were immunized s.c. four times using a γ2-GST fusion protein as antigen. The antigen contained 177 amino acid residues (res. # 391-567) from domain III of the γ2 (SEQ ID NO:8) (Kallunki, P., Sainio, K., Eddy, R., Byers, M., Kallunki, T., Sariola, H., Beck, K., Hirvonen, H., Shows, T. B. & Tryggvason, K. (1992) J. Cell Biol. 119: 679-693). Antibodies against the GST-epitopes were removed from the antisera by negative immunoadsorption with GST-Sepharose made by coupling E. coli expressed GST protein to CNBr-activated Sepharore. The removal of anti-GST IgG was ensured by Western blotting analysis with GST-specific antibodies. The specificity of the antibody against the laminin γ2 chain was also tested by Western blotting as well as by ELISA.

Polyclonal antibody against the C-terminus of the laminin γ2 chain was produced in rabbits essentially as above for domain III using a γ2-GST fusion protein as antigen. The antigen contained 161 amino acids (res. # 1017-1178) from domain I/II of the γ2 chain and antibodies against the GST-epitopes were removed from the antisera by negative immunoadsorption with GST-Sepharose. The specificity of the antibody was tested by Western blotting and ELISA.

Polyclonal antiserum against laminin-1 was a kind gift of Dr. Foidart (University of Liege, Belgium). Normal rabbit serum was obtained prior to immunization from the rabbits used for immunization. IgG from the laminin-1 and laminin γ2 chain antisera, as well as from normal rabbit serum, was purified using Protein A Sepharose (Pharmacia, Uppsala, Sweden).

Cell Adhesion Assay

Microtiter plates (96 wells: Nunc, Copenhagen, Denmark) were coated with 100 μl/well of laminin-1 (10 μg/ml), laminin-5 (10 μg/ml), or recombinant laminin γ2 chain (10 μg/ml) in PBS or 50 M Tris-HCl, pH 7.4 by incubating the plates overnight at 4° C. Control wells were uncoated or coated with the same amounts of BSA. In some experiment the proteins were first denatured by dialysis overnight against 5 M urea, 50 mM Tris-HCl, pH 7.5 and then renatured by dialysis against 50 mM Tris-HCl, pH 7.5. Potential remaining active sites on the plates were blocked with 150 μl of 10 mg/ml BSA in PBS for 2 hours at room temperature. The wells were washed with PBS, and 100 ml of Eagle's MEM containing 5 mg/ml BSA was added. For the adhesion assays, KLN205 cells were detached from subconfluent cell culture dishes with trypsin-EDTA (0.25%-0.03%) and resuspended in Eagle's MEM/BSA (5 mg/ml) at a concentration of 2×10⁵ cells/ml and allowed to recover for 20 minutes at 37° C. A total of 20,000 cells were then added to each well and allowed to attach for an additional 90 minutes at 37° C. The extent of cell adhesion was determined by measuring color yields at 600 nm, following fixation with 3% paraformaldehyde and staining with 0.1% crystal violet. For inhibition assays with the anti-γ2 antibody, the substrate coated wells were incubated with 20 μg/ml of anti-γ2 chain IgG in PBS for 60 minutes prior to incubations with the cells.

Migration Assay

The effect of endogenous laminin-5 on migration of KLN205 cells was determined by using a modified Boyden chamber assay, as described by Hujanen and Terranova (Hujanen, E. & Terranova, V. P. (1985) Cancer Res. 45: 3517-3521), and the effect of exogenous laminin-5 by using a modified Transwell assay, as described by Pelletier, et al. (Pelletier, A. J., Kunicki, T. and Quaranta, V. (1996), J. Biol. Chem. 271:364).

The Boyden chamber assay was carried out as follows. Polycarbonate filters (pore size 10 μm, diameter 12 mm; Costar, Cambridge, Mass.) were coated with 2.5 μg of EHS type IV collagen, and used to separate the upper and lower compartments of the 50 μl chamber. A total of 1×10⁵ cells in Eagle's MEM containing 0.1% BSA were placed in the upper compartment, and the lower compartment was filled with medium with or without chemoattractants (50 μg/ml laminin-1 or fibronectin). To study the effect of the laminin γ2 chain antibodies on cell migration, anti-γ2 (III) IgG or anti-γ2 (C-term) IgG was added to the upper compartment together with the cells at a concentration of 20 μg/ml. Normal rabbit IgG was used as a negative control. After an 8-hour incubation at 37° C. in a humidified atmosphere, the filters were removed, fixed and stained (Diff-Quick, Baxter Diagnostics, Tubingen, Germany). The cells that had not migrated were removed from the upper surface of the filter with cotton swabs. Migration of cells was quantified by counting the cells on the lower surface of each filter in 10 randomly selected high power fields (×400). All assays were performed in triplicate.

The “Transwell” plate assay (Transwell plates with pore size 12 μm, diameter 12 mm; Costar, Cambridge, Mass.) was used to determine the effect of exogenous laminin-5 on cell migration. The lower side of the membrane was coated with 2.5 μg of EHS type IV collagen for 3 hours at room temperature. Both sides were blocked with 1% bovine serum albumin for 1 hour. A total of 1×10⁵ cells were added per well in the upper compartment in Eagle's MEM containing 10% FCS, and the lower compartment was filled with 2.5 μg/ml laminin-5 as a chemoattractant. Antibodies against the C-terminus and domain III of the γ2 chains or nonimmune IgG were added to the upper compartment, together with the cells at a concentration 20 μg/ml. Following a 16-hour incubation at 37° C. the cells were fixed and stained. Cells on the top surface of the membrane were removed with cotton swabs, and cells that had migrated to the lower side of the membrane were counted (12 fields+/−S.D.).

Immunohistochemical Staining

Five μm thick paraffin sections were stained with polyclonal antibodies against laminin-1 or the γ2 chain of laminin-5. In brief, the paraffin sections were first incubated with 0.4% pepsin in 0.1 M HCl at 37° C. for 20 minutes to expose the antigens, blocked for nonspecific binding with 5% newborn rabbit serum, 0.1% BSA, and then incubated for 1 hour at 37° C. with the polyclonal IgG diluted in TBS to 5-10 μg/ml. Subsequently, a biotinylated swine-anti-rabbit antibody was applied, followed by incubation with a 1:400 dilution of Horseradish-Peroxidase-Avidin-Biotin-Complex (DAKO, Copenhagen, Denmark). The color was developed in diaminobentsamidine (DAB), followed by counterstaining of the slides with hematoxylin.

Results

Characterization of Proteins and Epithelium-Derived Cells

Immunopurified trimeric laminin-5, isolated from the culture medium of HaCat cells contained two major bands when analyzed by SDS-PAGE. These bands corresponded, respectively, to the 165 kDa γ2 chain, and the 155 kDa and 140 kDa γ2 and β3 chains migrating as a single band, as reported previously. Additionally, a weak band of about 105 kDa corresponding to the processed γ2 chain could be observed.

Full-length human recombinant laminin γ2 chain was produced in High-5 Spodoptera frugiperda insect cells using the baculovirus system. Since the γ2 chain was not secreted to the culture medium, possibly because it was not assembled intracellularly into a normal heterotrimer, it was isolated from the cell fraction as described in Materials and Methods. The protein was extracted under denaturating conditions using 5 M urea, renatured by extensive dialysis against 50 mM Tris-HCl, 100 mM NaCl, pH 7.4, and purified. The purified recombinant γ2 chain was full length (approximately 155 kDa) and highly pure as determined by SDS-PAGE analysis.

The HaCat human keratinocytes and mouse KLN205 squamous carcinoma cells were shown to express laminin-5, based on Northern blot analyses and immunostaining, using a cDNA probe and/or polyclonal antibodies specific for the γ2 chain, respectively. Furthermore, the KLN205 cells developed γ2 chain positive primary tumors and metasases in mice in vivo (data not shown). Following intramuscular or subcutaneous inoculations, large primary tumors developed in 4 weeks with numerous lung metastases after 4-6 weeks. KLN205 cells injected into the tail vein produced multiple lung tumors (experimental metastases) in four weeks. Consequently, both cell types were considered appropriate for the cell attachment and migration experiments carried out in this study.

Laminin-5 Molecule, but not Recombinant Laminin γ2 Chain, Promotes Cell Adhesion

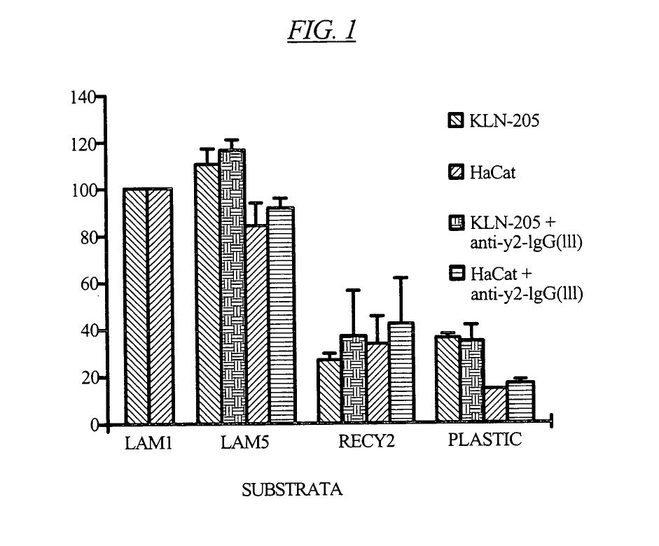

The laminin-5 and recombinant γ2 chain prepared in this study, as well as commercial laminin-1, were used as substrata in attachment assays (FIG. 1) with the two epithelium-derived HaCat and KLN205 cell lines that both express laminin-5. Both cell lines attached about 2.5 times more readily to laminin-1 than to plastic. Adhesion of the cells to laminin-5 appeared to be slightly higher than that to laminin-1, but the differences were not statistically significant. The cells attached equally well to laminin-5 preparations denatured in 5 M urea and then renatured by dialysis against 50 mM Tris-HCl, 100 mM NaCl, pH 7.4, as described for the recombinant γ2 chain above, indicating that this treatment did not affect the binding properties of the trimeric molecule. The attachment to laminin-5 did not significantly decrease in the presence of two different polyclonal antibodies made against the short or long arms of the γ2 chain or pre-IgG. Different amounts of the antibody against the short arm of the γ2 chain were also tested (up to 50 μg/ml), but no effects on cell adhesion were observed. When the cells were plated on the recombinant γ2 chain alone, the attachment was not significantly higher than that to plastic, this attachment not being influenced by polyclonal antibodies against the γ2 chain. The data confirm previous results showing that trimeric laminin-5 promotes adhesion of epithelial cells, but the present results further strongly suggest that this adhesion is not mediated by the γ2 chain.

Antibodies Against Laminin γ2 Domain III, but not Domain I/II, Inhibit Cell Migration

The potential role of the γ2 chain of laminin-5 in cell migration was examined for the KLN205 cells in vitro using Boyden and Transwell chamber assays as described in Materials and Methods.

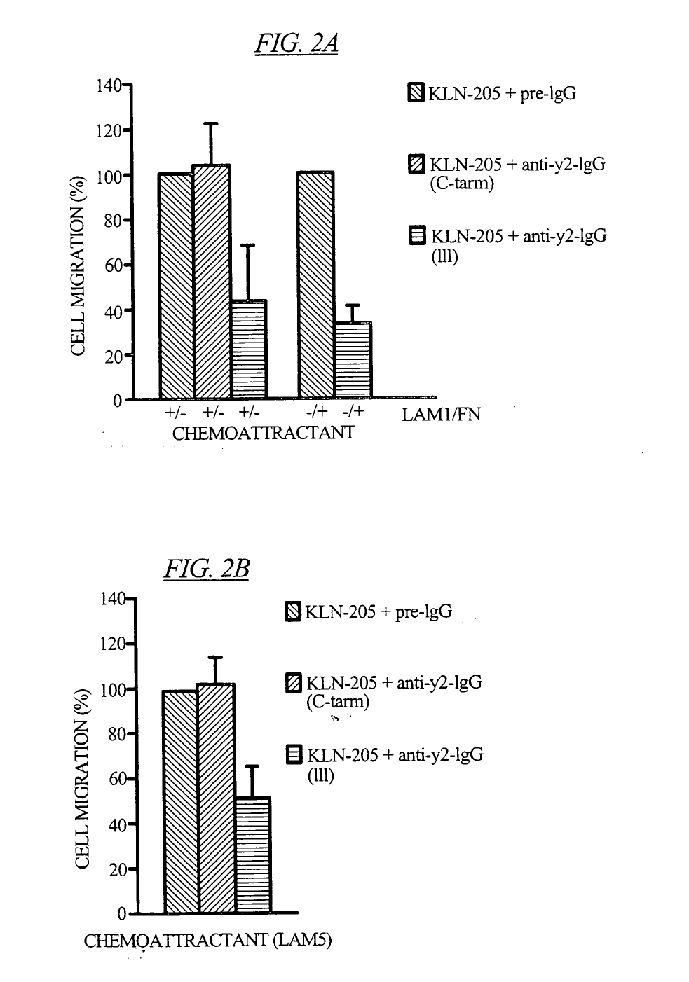

Migration was first studied in the Boyden chamber assay using laminin-1 and fibronectin in the lower chamber as chemoattractants (See FIG. 2A). The two compartments of the chemotacetic Boyden chambers were separated by a type IV collagen coated porous filter (pore size 8 μm). The cells (1×10⁵) in MEM containing 0.1% BSA were placed in the upper compartment, and laminin-1 (+/−) or fibronectin (−/+) in MEM containing 0.1% BSA were added as chemoattractants to the lower compartment. IgG against γ2 chain domains III, I/II or preimmune IgG was added to the upper compartment with the cells at a concentration of 20 μg/ml. After an 8-hour incubation at 37° C. the filters were removed and migration of cells to the lower surface of the filter was quantitated. The data are expressed as percentage of migrated cells (+/−SD (bars)) per high power field, setting migration in the presence of pre-immune IgG as 100%. Cells were counted in ten randomly selected high power fields to triplicate assays. When polyclonal IgG against the short arm of the γ2 chain was added to the upper compartment containing the cells, the migration of cells through the filter was decreased to about 35 to 45% of that observed with the preimmune serum. In contrast, the polyclonal IgG against C-terminal domain I/II did not affect migration of the cells.

The effects of the two antibodies were similarly used in the Transwell assay using native laminin-5 as chemoattractant in the lower compartment (See FIG. 2B). The lower side of the membrane was coated with EHS type IV collagen, and the lower compartment was filled with 2.5 μg/ml laminin-5 as a chemoattractant. Pre-immune IgG, IgG against the γ2 chain domains III or I/II were added to the upper chamber containing the cells. Following a 16-hour incubation the cells were fixed and cells at the lower side of the membrane were counted (12 fields+/−SD). The results were essentially the same as in the Boyden chamber assay. Thus, addition of IgG raised against domain III of the γ2 chain inhibited the migration to about 50% as compared with preimmune IgG, while the polyclonal IgG against domain I/II did not affect the cell migration.

These in vitro results demonstrate that laminin-5 have a role in the locomotion of epithelium-derived cells, and that this function can be inhibited by antibodies directed against domain III of the γ2 chain.

Thus, antibodies against the short arm of the laminin λ2 chain inhibited the migration of KLN205 squamous carcinoma cells by about 55-65% as determined in the Boyden chamber migration assay. Interestingly, the antibodies used here were directed against 177 amino acid residues of domain III (SEQ ID NO:8) that when deleted by mutation cause lethal junctional epidermolysis bullosa. Accordingly, the short arm of the laminin λ2 chain is important for the interaction of this laminin isoform to other extracellular matrix proteins and this interaction is also involved in the cell migration process.

### EXAMPLE 2

The following example describes, in detail, the preparation of monoclonal antibodies according to the invention as well as demonstrating their use in inhibiting tumor cell growth in laminin-5 secreting tumors.

Monoclonal antibodies against the γ2 chain of laminin-5 were produced by immunizing Balb/c mice with 100 ug GST-laminin-γ2-III fusion protein as antigen. The GST-laminin-γ2-III fusion protein contains human laminin-γ2-chain amino acid residues 391-567 (SEQ ID NO:8). Subsequent to immunization, spleen cells from the immunized mice were fused with mouse myeloma cell obtained from cell line P3X63Ag.8.653 (ATCC #CRL-1580). The hybridoma clones were then screened in immunohistology on frozen and paraffin sections (human cervix carcinoma, normal cervix and normal skin) for the production of the anti-laminin-γ2 antibody. The staining result was compared to negative control, mouse normal serum and IgG, and to the positive result obtained with well-characterized anti-laminin-5, γ2 chain polyclonal antibody (described in Pyke, et al., 1995). The hybridoma clones were also screened in ELISA. The best hybridoma clones were picked and cloned again twice (single cell cloning) to ensure that the produced hybridoma cell line was monoclonal.

The following describes the details of the production of three specific hybridoma clones and corresponding monoclonal antibodies produced therefrom. Characterization studies were conducted with respect to the 4G1, 5D5 and 6C12 monoclonal antibodies. Western blot analysis and ELISAs were carried out to confirm the specificity of the antibodies to the γ2 chain of laminin 5. Western blot analysis involved running recombinant laminin 5 γ2 chain (as well as appropriate controls) in an SDS-PAGE gel, blotting the gel on a nylon membrane, and incubating the membrane with the antibodies

For ELISA, plates were coated with 100 μl GST-γ2-III fusion protein (antigen) (Salo et al., Matrix Biology 18:197-210 (1999) at a concentration of 2.5 ug/ml in 0.1M carbonate/bi-carbonate buffer (pH 9) overnight at 4° C. (0.25 ug/well). The ELISA plate was then washed three times with a PBST solution (200 μl) (10 mM potassium phosphate, 150 mM NaCl), pH 7.5, and 0.05% Tween-20. Non-specific binding was then blocked by addition of BSA-PBS (1% bovine serum in PBS buffer (10 mM K₃P0₄, 150 mM NaCl, pH 7.5)) (200 m/well) for a period of 90 minutes. To this, a dilution of negative controls (normal mouse serum) and a sample diluted in BSA-PBS (Mab 4G1, 5D5 or 6C12) were added and then the ELISA plate was incubated for 1 hour at room temperature. After incubation, the ELISA plate was then washed with PBST three times. Next, HRP-conjugated anti-mouse IgG secondary antibody (Peroxidase (HRP) conjugated Rabbit Anti-Mouse IgG (H+L), Jackson Laboratories #315-035-045) was added and the plate was incubated at room temperature for 30 minutes. The ELISA plate was then washed again three times with PBST solution (200 ml). An ABTS-peroxide substrate was then added to the wells (ABTS diluted in 0.1 M Na-citrate, pH5; diluted immediately before assay use 1 ml to 10 ml with Na citrate buffer+2 μl 30% hydrogen peroxide) and then the plate was allowed to incubate in the dark for 30 minutes. The absorbance was then read with a micro plate reader at 405 nm at 30 and 60 minutes.

These analyses demonstrated the specificity of the monoclonal antibodies for domain III of the laminin 5 γ2 chain. Epitope mapping of the epitopes recognized by Mab 4G1, 5D5 or 6C12 indicated that they each bound epitopes within the amino acid sequence of SEQ ID NO:6 (which is a portion of domain III of the γ2 chain that lacks part of the amino and carboxy terminal portions of domain III), and did not bind to epitopes within the amino acid sequence of SEQ ID NOS: 9 and 10 (the 9 amino terminal and 41 carboxy terminal amino acids of domain III, respectively).

Monoclonal antibodies against the γ2 chain of laminin-5 were then tested for efficacy in inhibiting tumor cell growth in laminin-5 secreting tumors.

Study 1: Tumor Growth in Immunosuppressed Mice

The following study demonstrates the ability of IgG immunogloben against human laminin-5, γ2-III-domain (Mab 5D5) to affect the number and size of metastases in immune deficient mice.

10⁶ human squamous epithelial carcinoma cells were injected into the tail vein of immunosuppressed mice for tumor implantation. The cell lines used were human squamous epithelial carcinoma cells, cell line A431 and HSC-3. The cells were provided in suspensions in a medium containing DMEM-glutamax, 1% penicillin-streptomycin, 1% Na-pyruvate, 5% FCS. The cells were re-suspended in sterile Ca and Mg free PBS for inoculation. A control cell count was performed for the cell suspension at arrival and the cell density and the injected volume was recorded. The origin of the cells is HSC-3: Japan Health Science Research Resources Bank, JCRB 0623 A431: ATCC catalog number CRL-1555. The immunosuppressed mice were selected as they are susceptible to grow cells of human origin as is well known in the field. The tumor cells in groups 3 and 6 were injected into mice with test item (test item was 50 μg/ml) for tumor implantation. The tumor cells were allowed to grow for one week after which the animal received intravenous injections of the test item twice a week for four weeks.

TABLE 1
Study Layout

| | | | Animal | Cell | |
|---|---|---|---|---|---|
| Group | Mouse Strain | | Number | Line | Treatment |
| 1 | Balb/c˜nudet | 5 | 1-5 | — | −control, |
| | | | | | no treatment |
| 2 | Balb/c˜nude¹ | 5 | 6-10 | HSC-3 | +control, |
| | | | | | no treatment |
| 3 | Balb/c˜nude¹ | 5 | 11-15 | HSC-3 | Test item |
| | | | | | treatment: |
| | | | | | 50 μg 5D5l/mouse |
| | | | | | injection |
| 4 | SCID² | 5 | 21-25 | — | −control, |
| | | | | | no treatment |
| 5 | SCID˜ | 5 | 26-30 | A431 | +control, |
| | | | | | no test item |
| 6 | SCIDZ | 5 | 31-35 | A431 | Test item |
| | | | | | treatment: |
| | | | | | 50 μg 5D5/mouse |
| | | | | | injection |

¹Balb/c-nude (BALBicABom-nu, M&B A/S, Denmark)
²Fox Chase Scid (C.B-17/Icr scid/scid, M&B A/S, Denmark) immunodeficient mice.

After the treatment period, the animals were killed and tissue samples were collected. Number and size of the tumors in different tissues were counted and compared.

Test Items and Dosing Solutions

The test item was IgG immunoglobin against human laminin-5, γ2-III-domain (Mab 5D5). The test item was produced with monoclonal hybridoma method in vitro as set forth above. The test item (Mab 5D5) was suspended in sterile phosphate buffered saline (PBS) with a concentration of 1 mg/ml. The vehicle was sterilized using a 0.2 um filter. The delivered test item was diluted with sterile PBS 50:50 to give a dosing concentration of 500 μg/ml.

The test item was administered intravenously into the lateral tail vein of the immunosuppressed mice in a volume of 0.1 ml/animal. The dosing was twice a week on Mondays and Thursdays. The first dose of test item was administered one week after the induction of experimental metastasis.

After four weeks of treatment (eight doses of test item), the animals were killed by exsanguination with cardiac puncture in CO₂ anesthesia. Blood was collected and serum separated and frozen in −20° C. A gross necropsy was performed and the macroscopic signs were recorded with special attention to macroscopic tumor masses, which were calculated and measured if possible. The following organs/tissues were collected and weighed: lungs, lymph nodes (cervical and mesenerial), liver, and spleen. The organs/tissues were rinsed in PBS and fixed in 4% phosphate buffered formalin.

Clinical Signs

Animal number 6 had a thickening of the tail from day 5 through the whole study. The tail of animal number 11 turned dark/black after tumor cell inoculation and eventually turned necrotic. Half of the tail was missing from day 7 onward. No other treatment related clinical signs were recorded. One animal (number 8, group 2) was found dead on the morning of the day following tumor cell inoculation. Gross necropsy did not reveal any macroscopic changes. All other animals survived in good condition during the whole study.

Necropsy

The injected tumor cells induced tumor growth almost only in the lungs. Other tissues with macroscopic metastases include spleen, liver, small intestine, and preputial gland. The SCID mice had changes in the liver which might be of microbial origin. In the lungs, the metastases were so numerous and so small that it was impractical to calculate or measure individual metastases.

The following Table 2 represents a summarization of the results of the mice treated from Table 1.

TABLE 2
Experimental Metastases in Lung

| | | | | | Number of Mice |
|---|---|---|---|---|---|
| | | | | | with Macroscopic |
| | | | Cell | | Lung Metastases |
| Group | Mouse Strain | N | Line | Treatment | Observed |
| I | Balb/c-nude | 5 | — | −control, | — |
| | | | | no treatment | |
| 2* | Balb/c-nude | 5 | HSC-3 | +control, | 4/4 (full of |
| | | | | no treatment | mastastases) |
| 3 | Balb/c-nude | 5 | HSC-3 | Test item | 1/5 |
| | | | | treatment | |
| 4 | SCID˜ | 5 | — | −control, | — |
| | | | | no treatment | |
| 5 | SCID | 5 | A431 | +control, | 3/5 |
| | | | | no test item | |
| 6 | SCID | 5′ | A431 | Test item | 4/5 |
| | | | | treatment | |

*one mouse was dead at the end of the second study

As can be seen from Table 2 above, the treated Balb/c-nude mice had 1 of 5 mice with macroscopic lung metastases while 4 of 4 untreated control Balb/c-nude mice had macroscopic lung metastases.

### EXAMPLE 3

Monoclonal antibody 5D5 was tested against HT29 carcinomas in a tumor growth inhibition assay. The assay compared immunotherapy with 75 and 25 μg/mouse 5D5, qod×15, to conventional chemotherapy with 100 mg/kg CPT-11 (irinotecan/Campostar), qwk×3.

Methods and Materials

Female nude athymic mice (Harlan) were 13 weeks of age on day 1 of the study. The animals were fed ad libitum water (reverse osmosis, 1 ppm Cl) and the NIH 31 Modified and Irradiated Lab Diet® consisting of 18.0% protein, 5.0% fat, and 5.0% fiber. Mice were housed in static microisolators on a 12-hour light cycle at 21-22° C. (70-72° F.) and 40%-60% humidity.

Tumor Implantation

An HT29 carcinoma fragment (1 mm³) was implanted subcutaneously in the flank region of each mouse. When the tumors reached a size ranging from 62.5-126 mg, the mice were sorted into five treatment groups to provide a group mean tumor weights of 84.2-85.5 mg. Estimated tumor weight was calculated using the formula:

$$Tumor Weight ( mg ) = \frac{{w}^{2} \times 1}{2}$$

Where w=width and l=length in mm of the HT29 carcinoma.

Dosing solutions of 5D5 and control IgG were prepared fresh daily by dilution with phosphate-buffered saline. CPT-11 (Pharmacia; 20 mg/mL) was diluted with saline on each day of dosing.

On day 1, mice were sorted into five groups of animals (n=10/group), and dosing was initiated according to the protocols listed in Table 3.

TABLE 3
Protocol Design for the HT29-e29 Study

| | | Treatment Regimen I | | | |
|---|---|---|---|---|---|
| Group | n | Agent | mg/kg | Route | Schedule |
| I | 10 | No | n/a | | |
| | | treatment | | | |
| 2 | 10 | CPT-11 | 100 | IP | Qwk × 3 |
| 3 | 10 | Control lgG | 75 ug/mouse | IV | Qod × 15 |
| 4 | 10 | 5D5 | 75 ug/mouse | IV | Qod × 15 |
| 5 | 10 | 5D5 | 25 ug/mouse | IV | Qod × 15˜ |

As a positive reference drug, CPT-11 was administered once per week for three weeks (qwk×3) in 100 mg/kg doses. CPT-11 was delivered i.p. in volumes of 0.2 ml/20 g body weight, which were body-weight adjusted. Doses of 5D5 or control mouse IgG were delivered intravenously in volumes of 0.2 mL/mouse. The antibody doses were not body-weight adjusted. Untreated Group I mice served as controls for the CPT-11 therapy. Group 3 mice received 15 μg/mouse doses of control IgG once daily on alternate days (qod×15). Mice in groups 4 and 5 received 75 and 25 ug/mouse doses of 5D5×15, respectively.

Endpoint

Efficacy was evaluated in a tumor growth inhibition assay. Tumors were measured twice weekly until the study was terminated on day 31. Each animal was then euthanized and its HT29 carcincoma was excised and weighed. Treatment may produce complete tumor regression (CR) or partial tumor regression (PR) in an animal. In a CR response, there is no measurable tumor mass at the completion of the study. In a PR response, the tumor weight is lower than the weight on day, but greater than 0 mg. All tumors that did not regress were included in the calculation of tumor growth inhibition.

The increase in tumor weight for each animal was calculated as the difference between the actual tumor weight at the end of the study and the calculated tumor weight on day 1. These values were used to calculate the group mean tumor weight increases. Tumor growth inhibition was calculated from the group mean tumor weight increases of treated and control mice by the following equation:

$$\% TGI = [ \sim - \frac{( MeanNetTum {orWeight}_{Treated} )}{MeanNet Turn or {Weight}_{control}} 1 \times 100 \%$$

Toxicity

The mice were weighed twice weekly until the end of the study. They were examined frequently for clinical signs of any adverse, drug-related side effects. Acceptable toxicity for cancer drugs in mice is defined by the NCI as a mean group weight loss of less than 20% during the test, and not more than one toxic death among ten treated animals.

Statistics and Graphical Analyses

The unpaired t-test and Mann-Whitney U-test (for analysis of means and medians, respectively) were used to determine the statistical significance of the difference in mean tumor weights for mice in a treatment group and mice in a control group. The two-tailed statistical analyses were conducted at P=0.05.

Results

Efficacy: Growth of HT29 Colon Carcinomas in Control Mice

Treatment protocols are listed in Table 3. Group I mice received no treatment and served as controls for CPT-11 and 5D5 therapy. Group 3 mice received fifteen 75 μg/mouse doses of irrelevant mouse IgG on alternate days (qod×15). Table 4 summarizes the results for all groups in the study. The mean values for actual day 31 tumor weights in untreated and IgG-treated mice are 640.0 and 696.2 mg, respectively.

TABLE 4
Treatment Response Summary for the HT29-e28 Study

| | | | | | | | | | | | Max. % | | |
|---|---|---|---|---|---|---|---|---|---|---|---|---|---|
| | | | | | | | Tumor | | Mean % | | BW | # Death^{a} | |
| | | Regimen 1 | | | | Final Tumor Weight | Growth | # | Tumor | # | Loss | TR | |
| Gp. | | Agent | mg/kg | Route | Schedule | Mean ± SEM (n) | Inhibition | CP | Decrease | CR | Day | TR | NTR |
| 1 | 10 | No | n/a | | | 640.0 ± 124.9 mg (10) | 0% | 0 | None | 0 | −0.4%; | 0 | 0 |
| | | treatment | | | | | | | | | Day 4 | | |
| 2 | 10 | CPT-11 | 100 | IP | Qwk × 3 | 447.9 ± 91.8 mg(10) | 34.7% | 0 | None | 0 | −5.8%: | 0 | 0 |
| | | | | | | | | | | | Day4 | | |
| 3 | 10 | Control lgg | 75 μg/ | IV | Qod × 15 | 696.2 ± 131.4 mg (10) | 0% | 0. | None | 0 | −3.6%; | 0 | 0 |
| | | | mouse | | | | | | | | Day 4 | | |
| 4 | 10 | 5D5 | 75 μg/ | IV | Qod × 15 | 543.5 ± 149.3 mg (8) | 17.8% | | None | 0 | −2.0%; | 0 | 0 |
| | | | mouse | | | | | | | | Day 4 | | |
| 5 | 10 | 5D5 | 25 μg/ | IV | Qod × 15 | 700.7 ± 116.1 mg(10)˜ | 0% | 0 | None | 0 | −1.2%; | 0 | 0 |
| | | | mouse | | | | | | | | Day4 | | |

Response of HT29 Xerographs to Intraperitoneal CPT-11 Therapy

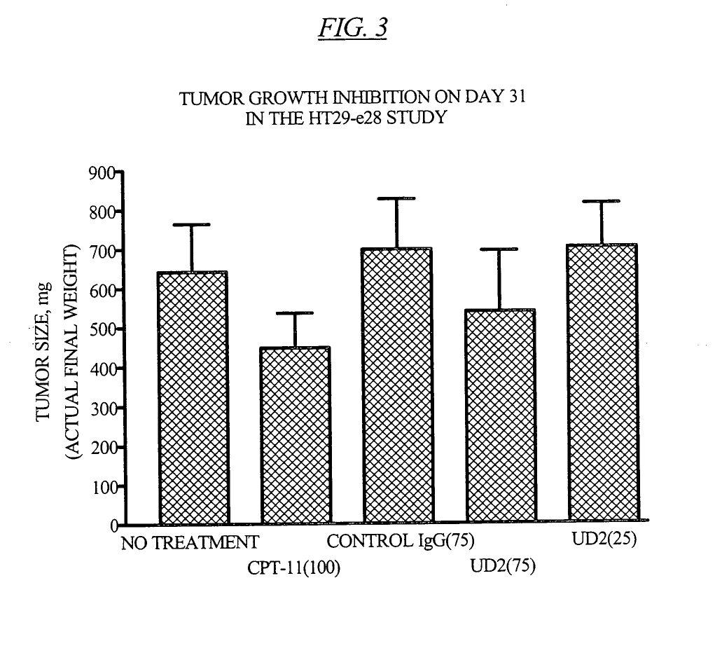

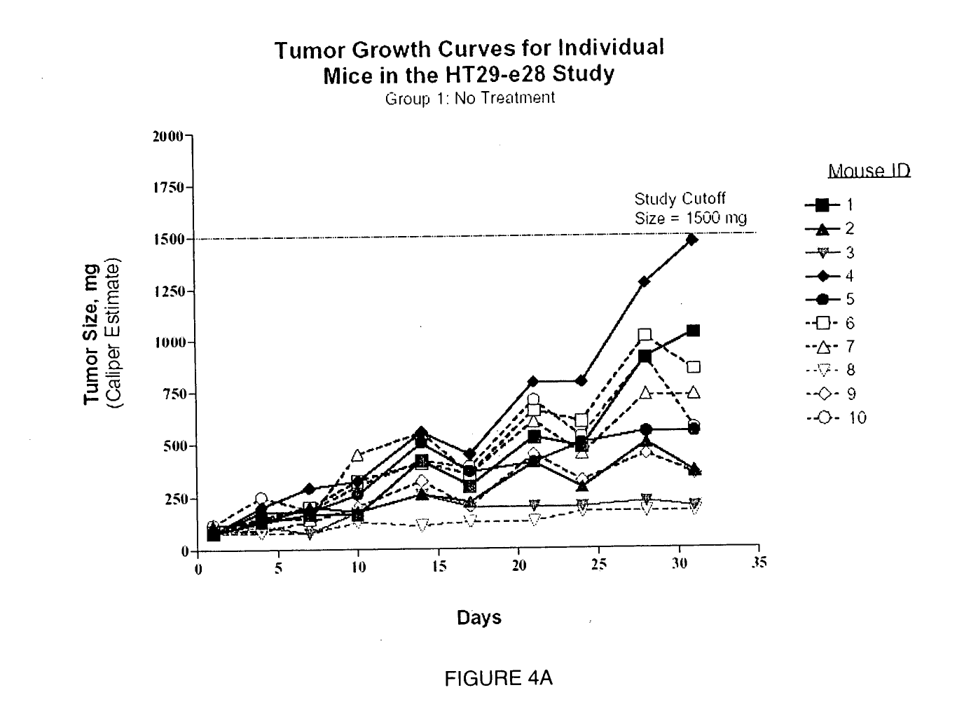

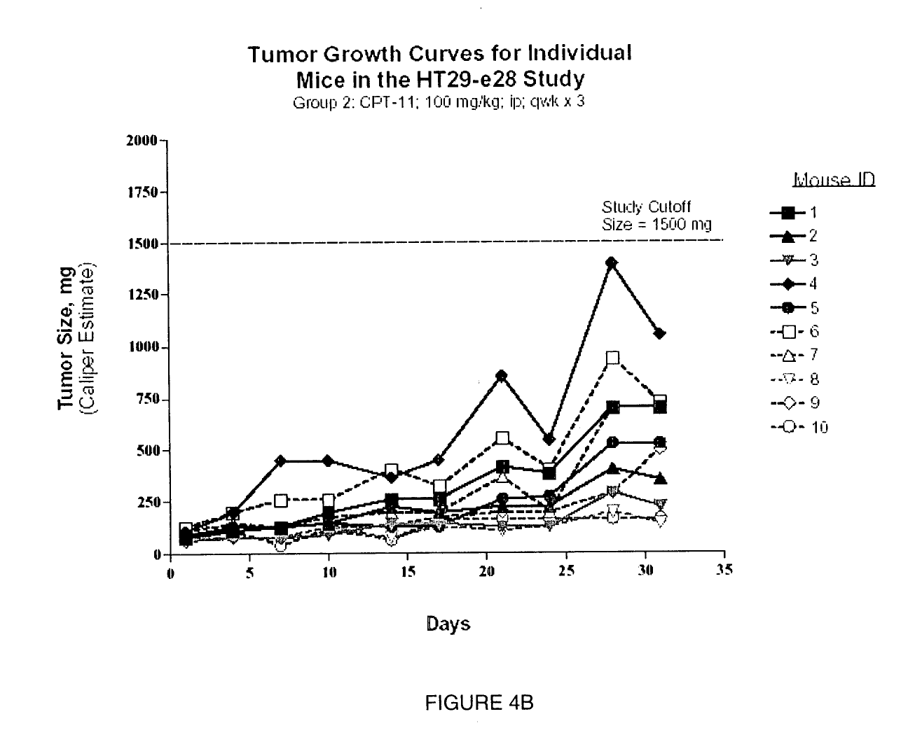

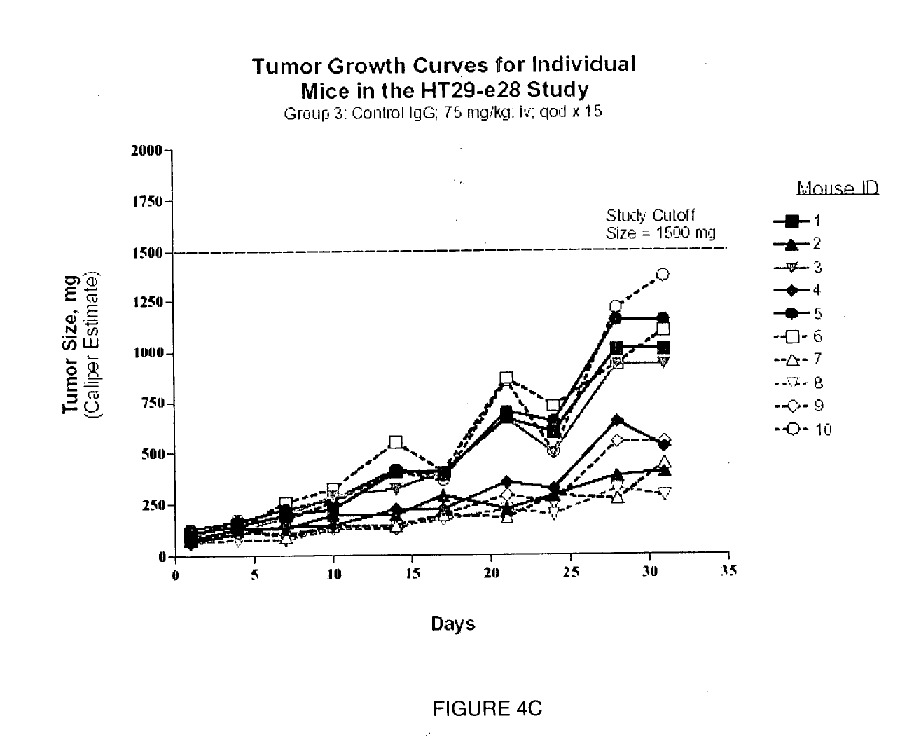

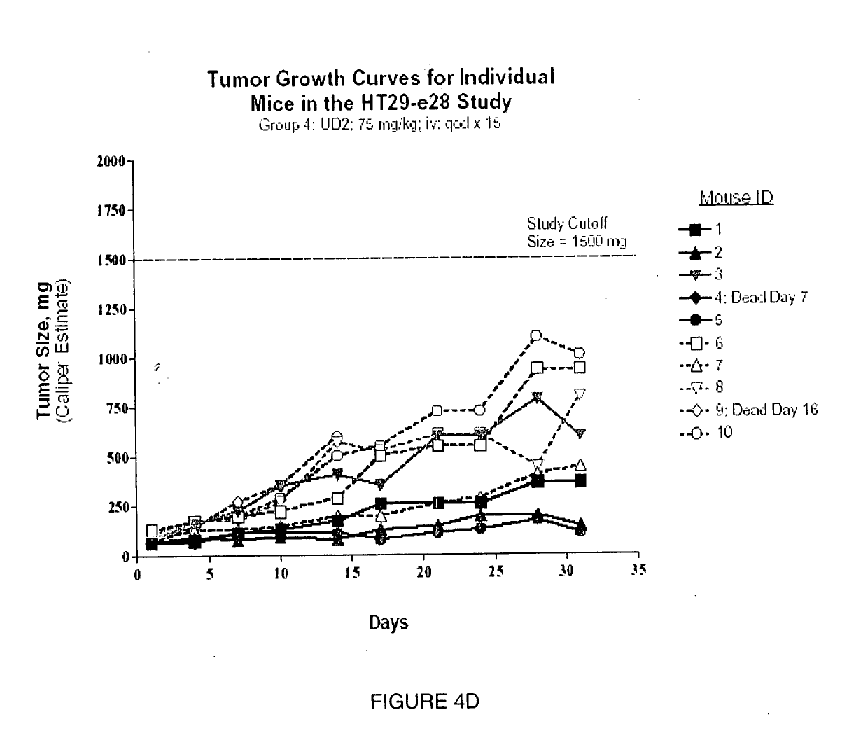

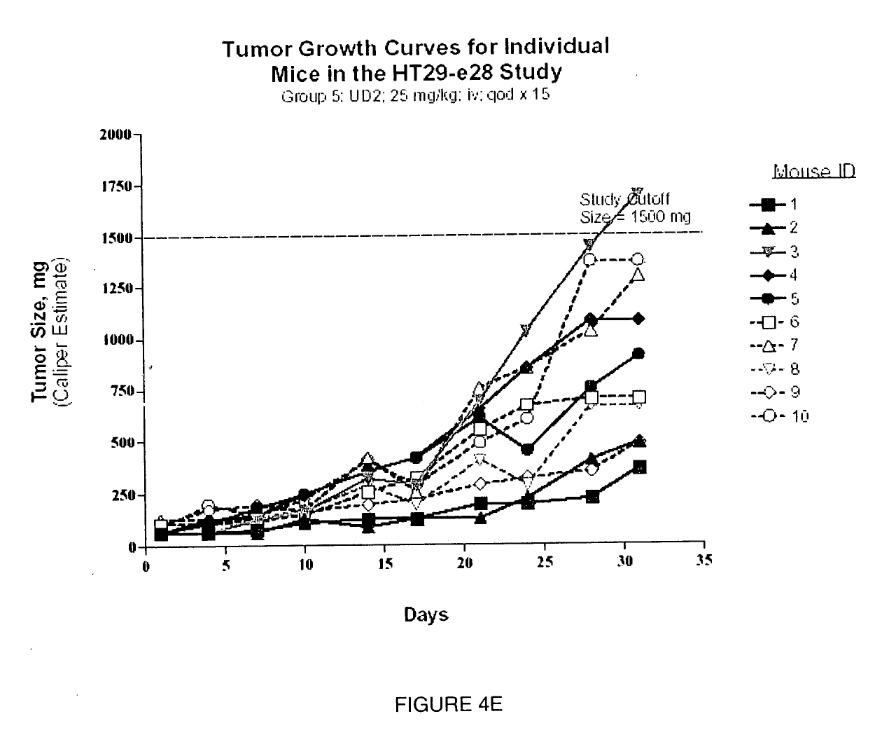

Group 2 mice were treated once weekly for three weeks (qwk×3) with i.p. injections' of 100 mg/kg CPT-11 (Table 3). No tumors regressed in response to CPT-11. The final mean tumor weight in Group 3 mice was 447.9 mg (Table 4). Group 2 mice experienced 34.7% tumor growth inhibition, relative to the untreated mice. This result, which is illustrated in a bar graph in FIG. 3, was not statistically significant (P=0.23.11, unpaired two-tailed t-test). FIGS. 4A-E shows the growth of individual tumors in all treatment groups, as calculated from caliper measurements. CPT-11 treatment caused a decrease in the slope of tumor growth.

Response of HT29 Xenografts to Intravenous 5D5 Immunotherapy

5D5 was administered intravenously to mice in Groups 4 and 5 on the qod×15 schedule at 75 and 25 ug/mouse, respectively (Table 3). No tumor regressions were observed. The 75 and 25 μg/mg mouse 5D5 treatments yielded final actual mean tumor weights of 543.5 and 700.7, respectively (Table 4). The high dose of 5D5 inhibited HT29 carcinoma growth by 17.8%, relative to tumor growth in untreated mice. Tumor growth inhibition in Group 4 mice, relative to untreated and IgG-treated mice, was not statistically significant (P=0.6241 and 0.453, respectively; t-test). Group 5 mice experienced no inhibition of tumor growth. FIG. 3 illustrates the lack of significant tumor growth inhibition, given the large error (SEM) bars. FIGS. 4 A-E shows that there was a modest decrease in the slopes of the tumor growth curves in animals treated with 75 ug/mouse 5D5.

Side Effects

All therapies were well tolerated. The highest group mean body-weight loss, an acceptable 5.8%, was recorded in mice treated with CPT-11. Body weight losses in antibody-treated mice were 3.6% or lower.

Discussion

The HT29 colon carcinoma xenograft model was appropriate for 5D5 evaluation because HT29 cells produce laminin. Growth of primary tumors can be impeded by anti-proliferative agents, such as CPT-11, as well as by agents that prevent invasion of the substratum. Combinational treatments using monoclonal antibodies against the γ2 chain of laminin-5, such as 5D5, with anti-proliferative agents such as CPT-11 are also contemplated as part of the invention. Treatment efficacy was based on tumor growth inhibition, i.e., the difference between the mean increase in tumor size in control and treated groups of animals during the 31-day study. Although there was no response to 5D5 at a dose of 25 μg/mouse, tumor growth was inhibited by 17.8% at 75 μg/mouse (Table 4 and FIG. 3). Thus, a 75 μg/mouse dose of 5D5 produced some therapeutic effect against HT29 colon carcinomas. In general, there was a reduction in the slopes of the tumor growth curves in mice treated with CPT-11 and 5D5 (FIGS. 4A-E). Accordingly, these results indicate that anti-laminin immunotherapy has application in cancer treatment of laminin-5 secreting tumors.

in summary, established HT29 colon carcinomas responded to therapy with 75 μg/mouse doses of 5D5. High dose 5D5 immunotherapy achieved 50% of the tumor growth inhibition that was produced by CPT-11 chemotherapy. The tumor growth shown in FIGS. 4A-E curves suggest that ′5D5 immunotherapy can impair colon tumor growth at doses of 75 pg/mouse or higher.

Those skilled in the art will know, or be able to ascertain, using no more than routine experimentation, many equivalents to the specific embodiments of the invention described herein. These and all other equivalents are intended to be encompassed by the following claims.

We claim:

1. A method for inhibiting tumor growth comprising administering to a subject with a laminin 5-secreting tumor an amount effective to inhibit tumor growth of an antibody that binds to one or more epitopes in domain III of laminin 5 γ2 chain.

2. The method of claim 1 wherein the antibody is a monoclonal antibody.

3. The method of claim 1 wherein the tumor is a carcinoma.

4. The method of claim 1, wherein the method further comprises treating the patient with surgery, chemotherapy, and/or radiation therapy.

5. An isolated antibody that binds to one or one or more epitopes of domain III of the laminin 5 γ2 chain but does not bind to epitopes within the amino acid sequence of SEQ ID NOS: 9 and 10.

6. The isolated antibody of claim 5 wherein the antibody is a monoclonal antibody.

7. A pharmaceutical composition comprising the isolated antibody of claim 5 and a pharmaceutically acceptable carrier.

8. Isolated hybridoma cells that express the monoclonal antibody of claim 6.

9. A pharmaceutical composition comprising an antibody that binds to one or more epitopes in domain III of laminin 5 γ2 chain and one or more further anti-tumor agent.

10. The pharmaceutical composition of claim 9 wherein the further anti-tumor agent is a chemotherapeutic.

11. A method for inhibiting tumor growth and/or metastasis comprising administering to a subject with a laminin 5-secreting tumor an amount effective to inhibit tumor growth and/or metastasis of the antibody of claim 5.

12. The method of claim 11 wherein the antibody is a monoclonal antibody.

13. The method of claim 11 wherein the tumor is a carcinoma.

14. The method of claim 11, wherein the method further comprises treating the patient with surgery, chemotherapy, and/or radiation therapy.

15. A method for detecting the presence of invasive cells in a tissue comprising contacting the tissue sample with one or more monoclonal antibody according to claim 5 to form an immunocomplex, and detecting formation of any immunocomplex, wherein the formation of the immunocomplex correlates with the presence of invasive cells in the tissue.
